(19) United States
(12) Patent Application Publication
(10) Pub. No.: US 2009/0211459 A1
(43) Pub. Date: Aug. 27, 2009

(54) DEODORIZATION DEVICE OF COOKING APPARATUS AND COOKING APPARATUS INCLUDING THE DEODORIZATION DEVICE

(75) Inventors: Jin Yul HU, Seoul (KR); Heung Shik CHOI, Seoul (KR); Hyun Jung KIM, Seoul (KR)

Correspondence Address: BIRCH STEWART KOLASCH & BIRCH, PO BOX 747, FALLS CHURCH, VA, 22040-0747, US

(73) Assignee: LG ELECTRONICS INC., Seoul, (KR)

(21) Appl. No.: 12/328,839

(22) Filed: Dec. 5, 2008

(30) Foreign Application Priority Data
Feb. 21, 2008 (KR) 10-2008-0015930

(51) Int. Cl. A47J 36/00; A61L 9/22

(52) U.S. Cl. 99/357; 422/120

(57) ABSTRACT

A deodorization device of a cooking apparatus and a cooking apparatus having the deodorization device are disclosed. Because a discharge space is formed between first and second discharging units and air containing an odorant material entirely passes through the discharge space, the odorant material in the discharge space can be effectively removed.

In addition, because the second discharging unit includes a plurality of electrode protrusions protrusively formed to be in contact with the first discharging unit, strong plasma can be generated in the vicinity of the electrode protrusions, so the deodorization effect can be improved.

Moreover, because the plurality of electrode protrusions and the first discharging unit are disposed to be separated from each other, a phenomenon that end portions of the electrode protrusions are abraded when high temperature plasma is generated can be minimized, and thus, the life span can be lengthened.

### BACKGROUND OF THE INVENTION

1. Field of the Invention

The present invention relates to a deodorization device of a cooking apparatus and a cooking apparatus having the deodorization device, and more particularly, to a deodorization device of a cooking apparatus and a cooking apparatus having the deodorization device capable of removing an odorant material generated in a cooking space.

2. Description of the Related Art

When a user is cooking (i.e., making dishes) by using a cooking apparatus, even after cooked dishes are taken out of the cooking apparatus, an odorant material (that emits objectionable odors) permeates and remains in the interior of the cooking apparatus. In addition, the odorant material is bound to get out of the interior of the cooking apparatus, making the user feel unpleasant.

### SUMMARY OF THE INVENTION

Thus, an object of the present invention is to provide a deodorization device of a cooking apparatus capable of effectively removing odorant material discharged from a cooking space.

To achieve the above object, there is provided a deodorization device of a cooking apparatus including: a first discharging unit disposed in a flow path of air including an odorant material; and a second discharging unit forming a discharge space with the first discharging unit, wherein the second discharging unit includes a second discharge electrode which includes an electrode plate unit and a plurality of electrode protrusions protrusively formed from the electrode plate unit toward the discharge space.

Each end portion of the plurality of electrode protrusions may be disposed to be in contact with the first discharging unit.

The plurality of electrode protrusions may be disposed to be separated by a pre-set interval from the first discharging unit.

The first discharging unit may include a first discharge electrode and a dielectric disposed on a surface of the first discharge electrode facing the discharge space.

The plurality of electrode protrusions may be disposed to be separated in at least one direction of a forward/back direction and a left/right direction.

The plurality of electrode protrusions may be formed to have width which diminishes as it goes toward the first discharging unit.

The thickness of each end portion of the plurality of electrode protrusions may be within a certain range of the interval between the plurality of electrode protrusions.

The second discharging unit may include a plurality of through holes allowing air, which has passed through the discharge space, to be discharged therethrough.

The second discharging unit may include a plurality of through holes allowing air containing an odorant material to be introduced into the discharge space.

The deodorization device of a cooking apparatus may further include a case to support the first and second discharging units.

The first discharging unit may include the first discharge electrode, and the case may include a fixing unit to fix the first and second discharge electrodes and a dielectric unit integrally molded with the fixing unit and disposed in the discharge space such that the dielectric unit is in contact with the first discharge electrode.

To achieve the above object, there is also provided a cooking apparatus including the deodorization device as described above.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a sectional view showing an air flow path of a cooking apparatus with a deodorization device disposed therein according to a first embodiment of the present invention.

FIG. 2 is a perspective view of the deodorization device in FIG. 1.

FIG. 3 is a perspective view of a second discharging unit in FIG. 2.

FIG. 4 is a perspective view of a deodorization device according to a second embodiment of the present invention.

FIG. 5 is a perspective view of a second discharging unit in FIG. 4.

FIG. 6 is a sectional view showing an air flow path of a cooking apparatus with a deodorization device disposed therein according to a third embodiment of the present invention.

FIG. 7 is a perspective view of first and second discharging units in FIG. 6.

FIG. 8 is a sectional view showing an air flow path of a cooking apparatus with a deodorization device disposed therein according to a fourth embodiment of the present invention.

FIG. 9 is a perspective view of first and second discharging units in FIG. 8.

FIG. 10 is a sectional view showing a deodorization device according to a fifth embodiment of the present invention.

FIG. 11 is a sectional view showing an air flow path of a cooking apparatus with a deodorization device disposed therein according to a sixth embodiment of the present invention.

FIG. 12 is a sectional view taken along line A-A in FIG. 11.

FIG. 13 is a sectional view showing a deodorization device according to a seventh embodiment of the present invention.

FIG. 14 is a sectional view showing an air flow path of a cooking apparatus with a deodorization device disposed therein according to an eighth embodiment of the present invention.

FIG. 15 is a perspective view of the deodorization device in FIG. 14.

FIG. 16 is a graph showing the shapes of electrode protrusion unit in FIG. 14 and the strength of electric fields.

### DETAILED DESCRIPTION OF THE PREFERRED EMBODIMENT

The foregoing and other objects, features, aspects and advantages of the present invention will become more apparent from the following detailed description of the present invention when taken in conjunction with the accompanying drawings. The present invention may be implemented in various forms without being limited to the embodiments described hereinafter.

A cooking apparatus according to an embodiment of the present invention includes any apparatus so long as it can perform cooking such as a microwave oven or the like.

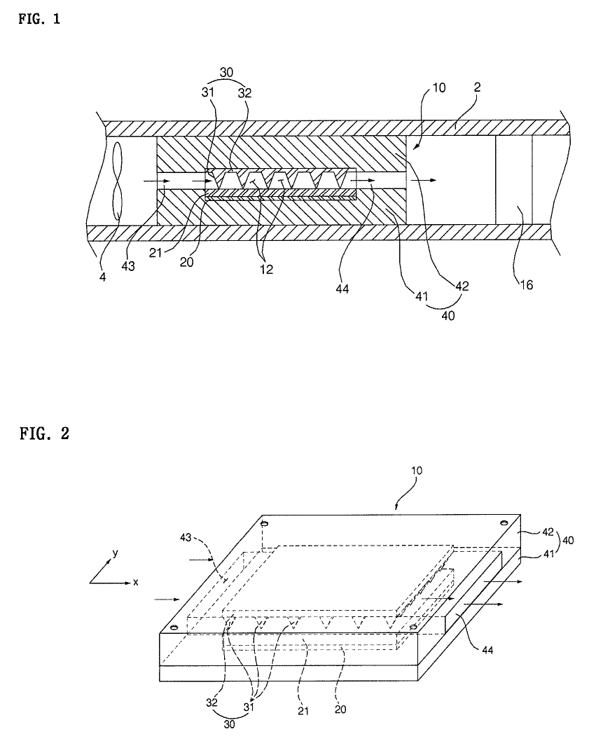

FIG. 1 is a sectional view showing an air flow path 2 of a cooking apparatus with a deodorization device 10 disposed therein according to a first embodiment of the present invention, and FIG. 2 is a perspective view of the deodorization device 10 in FIG. 1.

With reference to FIG. 1, the cooking apparatus includes: a cooking space (not shown) in which cooking is performed, and an air flow path 2 allowing air containing an odorant material discharged from the cooking space (not shown). The air flow path 2 is connected with the cooking space (not shown). The air flow path 2 may communicate with the exterior of the cooking apparatus. An air blower 4 may be disposed at the air flow path 2.

The cooking apparatus includes a deodorization device 10 to remove an odorant material of air that passes through the air flow path 2. The deodorization device 10 may be disposed at a rear side of the air blower 4 in the air flow path 2. The deodorization device 10 generates plasma to generate ozone and ions from air, and the generated ozone and ions removes the odorant material.

With reference to FIGS. 1 and 2, the deodorization device 10 includes the first discharging units 20 and 21, the second discharging unit 30 that forms a discharge space 12 with the first and second discharging units 20 and 21, and a case 40 supporting the first discharging units 20 and 21 and the second discharging unit 30.

The first discharging unit 20 and 21 refer to a first discharge electrode 20 and a dielectric 21 disposed on the first discharge electrode 20 facing the discharge space 12. The first discharge electrode 20 may be formed to be coated on the surface of the dielectric 21.

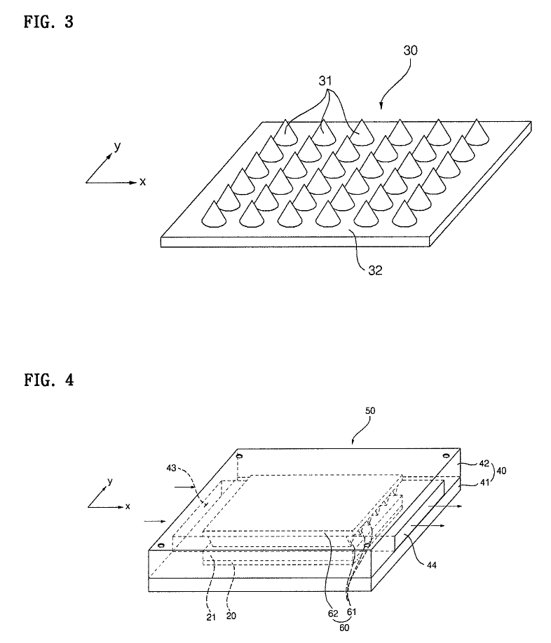

FIG. 3 is a perspective view of the second discharging unit in FIG. 2.

With reference to FIG. 3, the second discharging unit 30 refers to a second electrode. The second discharge electrode 30 includes a plurality of protrusions 31 protrusively formed toward the discharge space 12 and an electrode plate unit 32 supporting the plurality of electrode protrusions 31. The electrode plate unit 32 has a flat plate shape. The plurality of electrode protrusions 31 may be disposed to be spaced in a forward/backward direction or in a left/right direction on the electrode plate unit 32. Namely, the plurality of electrode protrusions 31 are formed in a pin shape and are separately disposed to be perpendicular to a direction in which air containing the odorant material flows. The plurality of electrode protrusions 31 are formed to have the width diminishing as it goes toward their end portions in the protruded direction. Namely, with reference to FIG. 3, the plurality of electrode protrusions 31 have a conical shape, but without being limited thereto, the plurality of electrode protrusions 31 may have the shape of trigonal pyramid, or may have tapered or rounded end portions. Accordingly, the contact area and time between the air containing the odorant material and the plurality of protrusions 31 can be increased to effectively remove the odorant material.

The respective end portions of the plurality of electrode protrusions 31 are disposed to be in contact with the dielectric 21. As the distance between the electrode protrusions 31 and the dielectric 21 becomes short, the strength of an electric field generated in the discharge space 12 is increased. The strength of plasma generated in the discharge space 12 is proportional to the strength of the electric field. Because the electrode protrusions 31 and the dielectric 21 are in contact with each other, stronger plasma can be generated in the discharge space 12.

The electrode protrusions 31 may be formed such that their widths diminish as it goes toward the dielectric 21. The thickness of the dielectric 21 may be smaller than the protruded length of the electrode protrusions 31.

The case 40 has the shape of a case for covering the first and second discharge electrodes 20 and 30. The first and second electrodes 20 and 30 may be simply disposed in the air flow path 2 by the case 40. The case 40 includes a first support 41 supporting the first discharge electrode 20 and a second support 42 combined with the first support 41 by a coupling member and supporting the second discharge electrode 30. The case 40 is formed of a dielectric. The second support 42 may include an inlet 43 to which a material containing an odor is introduced, and an outlet 44 from which deodorized air in the discharge space 12 is discharged. But the present invention is not limited thereto, and the inlet 43 and the outlet 44 may be formed at the first support 41 or may be formed at both the first support 41 and the second support 42. The inlet 43 and the outlet 44 are formed at a lower portion of the side of the second support 42 to allow air passing through the discharge space 12 to pass through the end portions of the plurality of electrode protrusions 31. The second support 42 may include a connection part (not shown) through which a voltage applying unit, an electric wire, or the like, may be connected to the second discharge electrode 30.

The first discharge electrode 20 is grounded, and a discharge voltage is applied to the second discharge electrode 30 by the voltage applying unit (not shown).

With reference to FIG. 1, an ozone removing unit 16 may be disposed at a rear side of the deodorization device 10 in the air flow path 12. The ozone removing unit 16 removes ozone remaining after being generated in the discharge space 12. The ozone removing unit 16 heads air introduced from the deodorization device 10 at higher than a set temperature to destroy ozone. However, without being limited thereto, the ozone removing unit 16 may remove ozone in various manners.

The operation of the deodorization device constructed as described above according to the first embodiment of the present invention will now be explained.

First, when cooking is performed in the cooking space (not shown), an odorant material is generated. In order to ventilate the cooking space, the air blower 4 operates. Then, air containing the odorant material in the cooking space passes through the air flow path 2. The air blown by the air blower 4 is introduced into the discharge space 12 via the inlet 43.

When voltage is applied to the second discharge electrode 30, plasma is generated in the discharge space 12. Ozone and ions are generated in the air within the discharge space 12 during plasma discharging. Various types of ions including anion such as hydroxyl ion or the like may be generated. The ions and ozone react to the odorant material introduced into the discharge space 12 to decompose and remove the odorant material.

Because the plurality of electrode protrusions 31 are disposed in the discharge space 12 and plasma is largely generated around the end portions of the electrode protrusions 31, air containing the odorant material can increasingly contact with plasma, compared with a case without such protrusions. Thus, the odorant material can be more effectively removed.

In addition, because the electrode protrusions 31 of the second discharge electrode 30 are in contact with the dielectric 21, strong plasma is generated between the electrode protrusions 31 and the dielectric 21 to improve the deodorization effect.

Also, because the discharge space 12 is limited by the first and second discharging units, air containing the odorant material passes all through the discharge space 12, so the odorant material can be removed.

Deodorized air (i.e., air without the odorant material) in the discharge space 12 is flows to the air flow path from the discharge space 12 via the outlet 44. The deodorized air may include ozone, which, thus, passes through the ozone removing unit 16. The ozone-removed air is externally discharged via the air flow path 2.

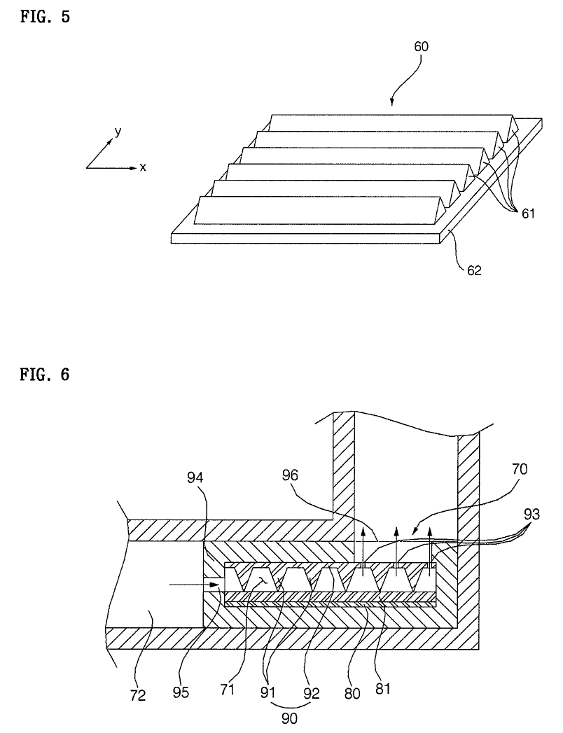

FIG. 4 is a perspective view of a deodorization device 50 according to a second embodiment of the present invention, and FIG. 5 is a perspective view of a second discharging unit in FIG. 4.

With reference to FIGS. 4 and 5, in the deodorization device 50 according to the second embodiment of the present invention, a second discharge electrode 60 includes a plurality of electrode protrusions 61 and an electrode plate unit 62 supporting the plurality of electrode protrusions 61, and a plurality of electrode protrusions 61 are formed long in a direction (x) along which the air containing the odorant material flows, and formed separately in a direction (y) perpendicular to the direction (x). Other construction and operation of the deodorization device 50 according to the second embodiment of the present invention may be implemented or performed in the same or similar manner as those of the deodorization device 10 according to the first embodiment of the present invention, so the repeated description will be omitted.

In the deodorization device according to the second embodiment of the present invention, the electrode protrusions 61 are formed long in the direction in which the air containing the odorant material flows. Accordingly, because air containing the odorant material continuously contacts with the plurality of electrode protrusions 61 while the air is being guided along by the electrode protrusions, the odorant material can be more effectively removed.

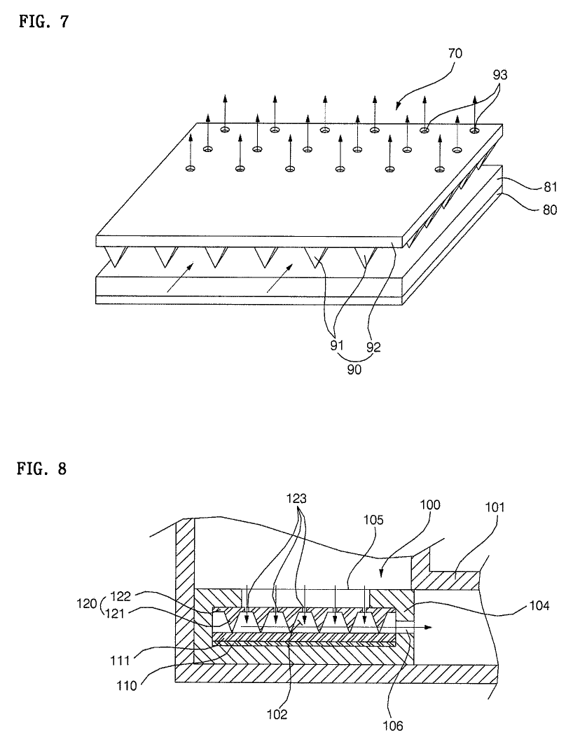

FIG. 6 is a sectional view showing an air flow path of a cooking apparatus with a deodorization device 70 disposed therein according to a third embodiment of the present invention, and FIG. 7 is a perspective view of first and second discharging units in FIG. 6.

As shown in FIGS. 6 and 7, the deodorization device 70 according to the third embodiment of the present invention includes a first discharging unit disposed in an air flow path 72, a second discharging unit forming a discharge space 71 with the first discharging unit, and a case supporting the first and second discharging units. The first discharging unit includes a first discharge electrode 80 and a dielectric 81, and the second discharging unit refers to a second discharge electrode 90. The second discharge electrode 90 includes a plurality of electrode protrusions 91 protrusively formed toward the discharge space 71, and an electrode plate unit 92 supporting the plurality of electrode protrusions 91. The second discharge electrode 90 includes a plurality of through holes 93 formed to allow odorant material-removed air to be discharged from the discharge space 71 therethrough. Other construction and operation of the deodorization device 70 according to the third embodiment of the present invention may be implemented or performed in the same or similar manner as those of the deodorization device 10 according to the first embodiment of the present invention, so the repeated description will be omitted.

The plurality of through holes 93 may be disposed between the plurality of electrode protrusions 91. In addition, in order to secure a time during which air containing an odorant material introduced into the discharge space 71 contacts with the electrode protrusions 91, the plurality of through holes 93 may be formed at a position spaced apart from the side where the air containing the odorant material is introduced. But without being limited thereto, the plurality of through holes 93 may be disposed on the entire surface of the electrode plate unit 92.

A case 94 includes an inlet 95 to which the air containing the odorant material is introduced, and an outlet 96 formed to correspond to the through holes 93 and allowing deodorized air to flow out therethrough.

In the deodorization device 70 according to the third embodiment of the present invention, the positions of the plurality of through holes 93 may be adjusted to control time during which the air containing the odorant material and the plurality of electrode protrusions 91 contact with each other. In addition, the deodorization device 70 may be installed at an air flow path that has a different shape other than the linear shape.

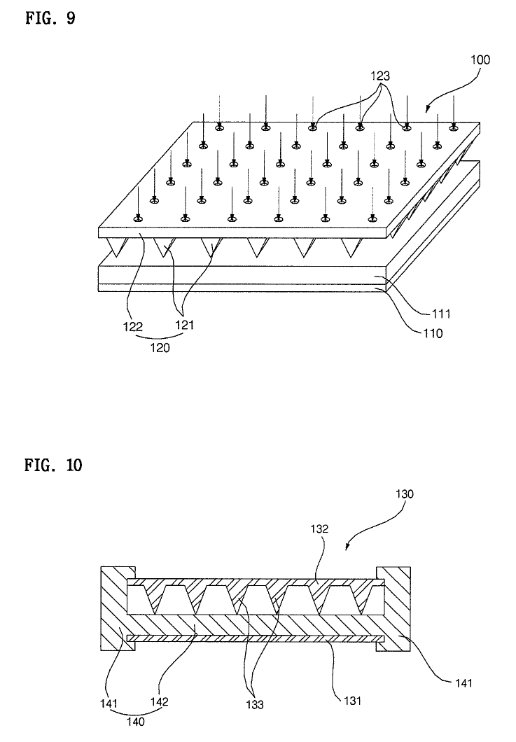

FIG. 8 is a sectional view showing an air flow path of a cooking apparatus with a deodorization device 100 disposed therein according to a fourth embodiment of the present invention, and FIG. 9 is a perspective view of first and second discharging units in FIG. 8.

As shown in FIGS. 8 and 9, the deodorization device 100 according to the fourth embodiment of the present invention includes a first discharging unit disposed in an air flow path 101, a second discharging unit forming a discharge space 102 with the first discharging unit, and a case supporting the first and second discharging units. The first discharging unit includes a first discharge electrode 110 and a dielectric 111, and the second discharging unit is a second discharge electrode 120. The second discharge electrode 120 includes a plurality of electrode protrusions 121 protrusively formed toward the discharge space 102 and an electrode plate unit 122 supporting the plurality of protrusions 121. The second discharge electrode 120 includes a plurality of through holes 123 to allow air containing an odorant material to be introduced therethrough. Other construction and operation of the deodorization device 100 according to the fourth embodiment of the present invention may be implemented or performed in the same or similar manner as those of the deodorization device 70 according to the third embodiment of the present invention, so the repeated description will be omitted.

An inlet 105 is formed on an upper surface of a case 104 to allow air containing an odorant material from the air flow path 101 to be introduced therethrough, and an outlet 106 is formed at the side of the case 104 to allow air without the odorant material as it has passed through the discharge space 102 to flow out therethrough. The single inlet 105 is formed at a position corresponding to the plurality of through holes 123. But without being limited thereto, a plurality of inlets may be formed such that they correspond to the plurality of through holes 123 respectively.

The deodorization device 100 according to the fourth embodiment of the present invention the air containing odorant material flows toward end portions of the plurality of electrode protrusions 121 via the inlet 105 and the plurality of through holes 123. Accordingly, the air largely comes in contact with plasma around the end portions of the plurality of electrode protrusions 121 where plasma is mainly generated, so the odorant material can be more effectively removed.

FIG. 10 is a sectional view showing a deodorization device 130 according to a fifth embodiment of the present invention.

With reference to FIG. 10, the deodorization device 130 according to the fifth embodiment of the present invention includes a first discharge electrode 131, a second discharge electrode 132 forming a discharge space with the first discharge electrode 131 and including a plurality of electrode protrusions 133, and a case 140 supporting the first and second discharge electrodes 131 and 132. The case 140 includes a fixing unit 141 for fixing the first and second discharge electrodes 131 and 132 and a dielectric unit 142 integrally molded with the fixing unit 141 and disposed to be in contact with the first discharge electrode 131. Other construction and operation of the deodorization device 130 according to the fifth embodiment of the present invention may be implemented or performed in the same or similar manner as those of the deodorization device 10 according to the first embodiment of the present invention, so the repeated description will be omitted.

Namely, the fixing unit 141 of the case 140 fixes the first and second discharge electrodes 131 and 132 by surrounding them, and the dielectric unit 142 of the case 140 is in contact with one surface of the first discharge electrode and disposed in the discharge space.

In the deodorization device 130 according to the fifth embodiment of the present invention constructed as described above, because the case 140 includes the dielectric unit 142, any other additional dielectric is not required, and thus, the structure can be simplified and the number of components can be reduced.

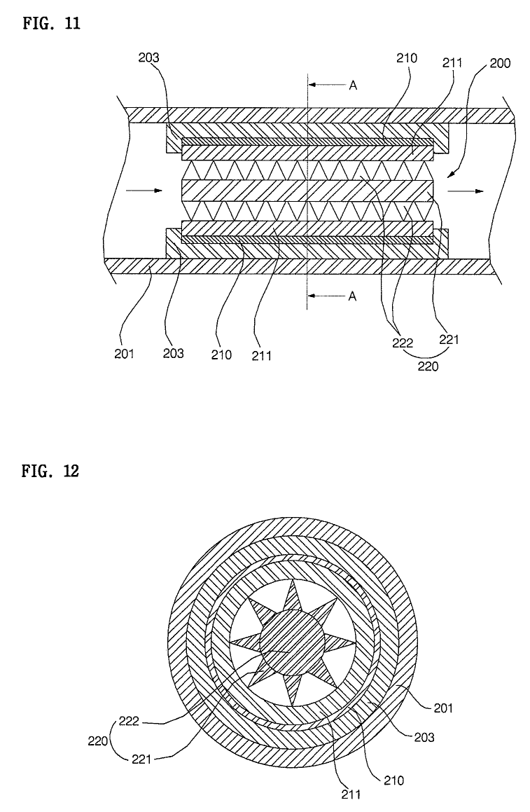

FIG. 11 is a sectional view showing an air flow path of a cooking apparatus with a deodorization device 200 disposed therein according to a sixth embodiment of the present invention, and FIG. 12 is a sectional view taken along line A-A in FIG. 11.

With reference to FIGS. 11 and 12, the deodorization device 200 according to the sixth embodiment of the present invention includes a first discharging unit disposed in an air flow path 201, a second discharging unit forming a discharge space with the first discharging unit, and a case 203 supporting the first and second discharging units. The first discharging unit includes an annular first discharge electrode 210 and a dielectric 211 disposed to be in contact with an inner circumferential surface of the first discharge electrode 210. The dielectric 211 also has a ring shape. The second discharging unit is a second discharge electrode 220 which includes an electrode column unit 221 formed in a columnar shape and a plurality of protrusions 222 protrusively formed toward the dielectric from an outer circumferential surface of the electrode column unit 221. Other construction and operation of the deodorization device 200 according to the sixth embodiment of the present invention may be implemented or performed in the same or similar manner as those of the deodorization device 10 according to the first embodiment of the present invention, so the repeated description will be omitted.

The electrode column unit 221 of the second discharge electrode 220 may be formed in a cylindrical shape. But without being limited thereto, the electrode column unit 221 may be formed in a polygonal columnar shape. The plurality of electrode protrusions 221 and the dielectric 211 are disposed to be in contact with each other. The plurality of electrode protrusions 222 are disposed to be separated at certain intervals in a lengthwise direction and in a circumferential direction of the electrode column unit 221, whereby the area in which the electrode protrusions 222 and air containing an odorant material contact with each other can be maximized.

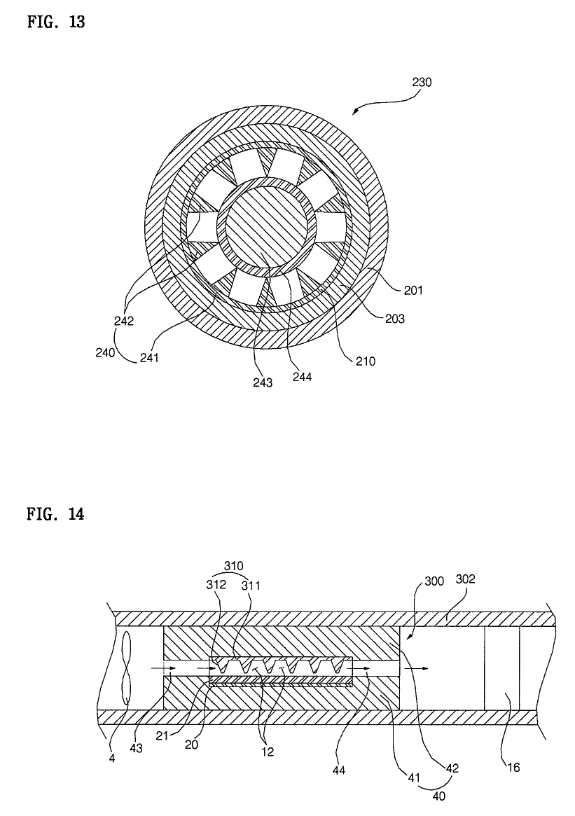

FIG. 13 is a sectional view showing a deodorization device 230 according to a seventh embodiment of the present invention.

With reference to FIG. 13, in the deodorization device 230 according to the seventh embodiment of the present invention, a second discharge electrode 240 includes an annular electrode ring unit 241 and a plurality of electrode protrusions 242 formed to be protruded from an inner circumferential surface of the electrode ring unit 241. A first discharge electrode 243 has a columnar shape. Other construction and operation of the deodorization device 230 according to the seventh embodiment of the present invention may be implemented or performed in the same or similar manner as those of the deodorization device 200 according to the sixth embodiment of the present invention, so the repeated description will be omitted. The electrode ring unit 241 is disposed to surround the first discharge electrode 243, and a dielectric 244 is disposed on an outer circumferential surface of the first discharge electrode 243 such that the dielectric 244 is in contact with the plurality of electrode protrusions 242. Because the plurality of electrode protrusions 242 are disposed to be separated at certain intervals in a lengthwise direction and in a circumferential direction of the electrode ring 241, the area in which air containing an odorant material and the electrode protrusions 242 contact with each other can be maximized.

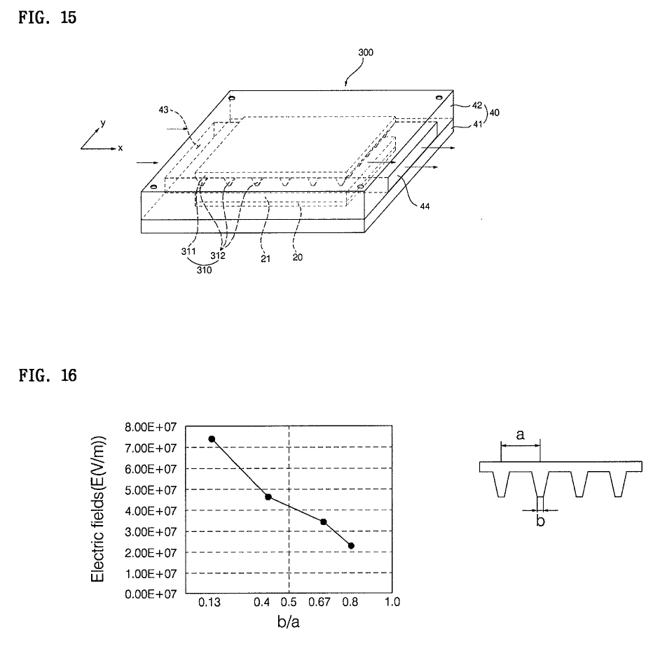

FIG. 14 is a sectional view showing an air flow path 302 of a cooking apparatus with a deodorization device 300 disposed therein according to an eighth embodiment of the present invention, and FIG. 15 is a perspective view of the deodorization device 300 in FIG. 14.

As shown in FIG. 14, in the deodorization device 300 according to eighth embodiment of the present invention, a second discharge electrode 310 includes an electrode plate unit 311 and electrode protrusions 312. Respective end portions of the electrode protrusions 312 are disposed to be separated at a set interval from a dielectric 21. Other construction and operation of the deodorization device 50 according to the second embodiment of the present invention may be implemented or performed in the same or similar manner as those of the deodorization device 10 according to the first embodiment of the present invention, so the repeated description will be omitted.

The interval between the electrode protrusions 312 and the dielectric 21 may be smaller than the set interval. As the distance between the electrode protrusions 312 and the dielectric 21 becomes shorter, the strength of plasma and electric field generated in the discharge space 12 is increased. Thus, the interval between the electrode protrusions 312 and the dielectric 21 may be previously set according to experimentation such that the strength of the electric field generated between the electrode protrusions 3132 and the dielectric 21 is greater than a certain value.

Because the plurality of electrode protrusions 312 and the dielectric 21 are not in contact with each other, abrasion of the electrode protrusions 312 that may be otherwise caused due to generation of high temperature plasma between the electrode protrusion 312 and the dielectric 21 can be minimized or prevented, and thus, the life span can be lengthened and the reliability can be improved.

FIG. 16 is a graph showing the shapes of electrode protrusion unit in FIG. 14 and the strength of electric fields.

With reference to FIG. 16, as end portions of the plurality of electrode protrusions 312 are sharper, the strength of the electric field is increased. Thus, the thickness of the respective electrode protrusion 312 diminishes as it goes toward their end portions in the protruded direction. Namely, the width (or thickness) (b) of each end portion of the electrode protrusions 312 may be within a certain range of the interval (a) between the electrode protrusions. In this embodiment, the width (b) of the end portion of each electrode protrusion 312 may be set to be within a half of the interval (a) between the electrode protrusions.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

The deodorization device of a cooking apparatus and the cooking apparatus having the deodorization device according to the present invention is advantageous in that because the discharge space is formed between the first and second discharging units and air containing an odorant material entirely passes through the discharge space, the odorant material in the discharge space can be effectively removed.

In addition, because the second discharging unit includes the plurality of electrode protrusions protrusively formed to be in contact with the first discharging unit, strong plasma can be generated in the vicinity of the electrode protrusions, so the deodorization effect can be improved.

Moreover, because the plurality of electrode protrusions and the first discharging unit are disposed to be separated from each other, the phenomenon that the end portions of the electrode protrusions are abraded when high temperature plasma is generated can be minimized, and thus, the life span can be lengthened.

What is claimed is:

1. A deodorization device for a cooking apparatus, comprising:
a first discharger; and
a second discharger, the first and second dischargers being disposed in a flow path of air containing odorant material, and forming a discharge space therebetween,
wherein the second discharger includes a second discharge electrode having an electrode plate portion and a plurality of electrode protrusions extending from the electrode plate portion toward the discharge space.

2. The device of claim 1, wherein end portions of the plurality of electrode protrusions contact the first discharger.

3. The device of claim 1, wherein the plurality of electrode protrusions are spaced from the first discharger by a predetermined amount.

4. The device of claim 1, wherein the first discharger comprises a first discharge electrode and a dielectric disposed on a surface of the first discharge electrode facing the discharge space.

5. The device of claim 4, wherein end portions of the plurality of electrode protrusions contact the dielectric.

6. The device of claim 1, wherein the plurality of electrode protrusions are spaced apart from each other on the electrode plate portion.

7. The device of claim 1, wherein the plurality of electrode protrusions are arranged in rows on the electrode plate portion.

8. The device of claim 1, wherein the plurality of electrode protrusions are elongated in a direction of air flow in the discharge space.

9. The device of claim 1, wherein widths of the plurality of electrode protrusions decrease toward the first discharger.

10. The device of claim 1, wherein the plurality of electrode protrusions are tapered toward the first discharger.

11. The device of claim 1, wherein a thickness of end portions of the plurality of electrode protrusions is within a predetermined range of an interval between adjacent electrode protrusions.

12. The device of claim 1, wherein the first discharger is grounded and the second discharger is supplied with voltage so that plasma discharge is generated in the discharge space.

13. The device of claim 1, wherein the second discharger comprises a plurality of through holes configured to pass air which has flowed through the discharge space.

14. The device of claim 1, wherein the second discharger comprises a plurality of through holes configured to pass air containing odorant material into the discharge space.

15. The device of claim 1, further comprising:
a case configured to support the first and second dischargers.

16. The device of claim 15, wherein the case comprises an inlet configured to pass air containing odorant material into the discharge space, and an outlet configured to pass air which has flowed through the discharge space.

17. The device of claim 15, wherein
the first discharger comprises a first discharge electrode, and
the case comprises a holding portion configured to hold the first and second discharge electrodes, and a dielectric portion integrally formed with the holding portion and disposed in the discharge space such that the dielectric portion contacts the first discharge electrode.

18. The device of claim 1, wherein the electrode plate portion is annular, and the plurality of electrode protrusions extend from an inner surface of the electrode plate portion.

19. The device of claim 18, wherein the first discharger is substantially cylindrical and is disposed inside of the second discharger.

20. The device of claim 19, wherein the first discharger comprises a substantially cylindrical first discharge electrode and an annular dielectric disposed on an outer surface of the first discharge electrode.

21. The device of claim 1, wherein the electrode plate portion is substantially cylindrical, and the plurality of electrode protrusions extend from an outer surface of the electrode plate portion.

22. The device of claim 21, wherein the first discharger is annular and is disposed around the second discharger.

23. The device of claim 22, wherein the first discharger comprises an annular first discharge electrode and an annular dielectric disposed on an inner surface of the first discharge electrode.

24. A cooking apparatus comprising the deodorization device of claim 1.

25. The apparatus of claim 24, further comprising an ozone remover.
